(19) United States
(12) Patent Application Publication
(10) Pub. No.: US 2011/0270146 A1
(43) Pub. Date: Nov. 3, 2011

(54) Orthotic

(75) Inventors: Liang-Wey Chang, Zhubei City (TW); Shun-Yi Yao, Banqiao City (TW); Hui-Chun Ho, Zhubei City (TW); Yee-Pien Yang, Taipei City (TW)

(73) Assignee: INDUSTRIAL TECHNOLOGY RESEARCH INSTITUTE, Hsinchu (TW)

(21) Appl. No.: 12/771,007

(22) Filed: Apr. 30, 2010

(51) Int. Cl. A61F 5/00

(52) U.S. Cl. 602/23

(57) ABSTRACT

An orthotic for correcting a movement of a hip joint of a user includes a support frame, a cam, a leg fixer, a following element and a restoring element. The support frame is attached to the user. The cam is disposed to the support frame. The leg fixer is pivotally connected to the support frame. The following element is moveably mounted on the leg fixer and connected to the cam. The restoring element connected to the leg fixer provides a force of restoring the leg fixer when the leg fixer is moved. When the leg fixer is being moved, a predetermined relative movement between the cam and the following element is produced to correct a movement range of the hip joint.

### BACKGROUND OF THE DISCLOSURE

1. Field of the Disclosure

The disclosure relates in general to an orthotic, and more particularly to an orthotic having a cam.

2. Description of the Related Art

A conventional orthotic for correcting a movement of a hip joint mainly includes a hinge mechanism, which can restrict the movement of the hip joint of the human body so that the hip joint only can perform the flexion/extension movement and is not allowed to perform the adduction/abduction movement.

However, a normal gait curve simultaneously includes movements of flexion, extension, abduction and adduction. The above-mentioned hinge mechanism only provides the degrees of freedom of some of the movements of flexion, extension, adduction and abduction, but cannot simultaneously provide the degrees of freedom of all the movements of flexion, extension, adduction and abduction. Thus, the patient cannot obtain the correct correction. In this case, the power of the patient is also consumed when the patient uses the conventional orthotic for a long time, thereby causing the great loading on the power of the patient.

### SUMMARY OF THE DISCLOSURE

The disclosure is directed to an orthotic for correcting a movement of a hip joint. The orthotic can simultaneously provide the degrees of freedom of the movements of flexion, extension, adduction and abduction in the walking friction of the patient so that the patient can obtain opportune correcting procedure closer to the normal gait curve.

According to the present disclosure, an orthotic for correcting a movement of a hip joint of a user is provided. The orthotic includes a support frame, a cam, a leg fixer, a following element and a restoring element. The support frame is attached to the user. The cam is disposed to the support frame. The leg fixer is pivotally connected to the support frame. The following element is moveably mounted on the leg fixer and contacted to the cam. The restoring element connected to the leg fixer provides a force of restoring the leg fixer when the leg fixer is moved. When the leg fixer is being moved, a predetermined relative movement between the cam and the following element is generated to correct a movement range of the hip joint.

The disclosure will become apparent from the following detailed description of the preferred but non-limiting embodiments. The following description is made with reference to the accompanying drawings.

### BRIEF DESCRIPTION OF THE DRAWINGS

FIG. 1 is an assembled view showing an orthotic according to a preferred embodiment of the disclosure.

FIG. 2 is a schematic illustration showing the orthotic of FIG. 1 attached to a patient.

FIG. 3 shows a normal gait curve.

FIG. 4 shows the coordinate definition of the gait curve of FIG. 3.

FIG. 5 shows the movement of flexion and extension.

FIG. 6 shows the movement of abduction and adduction.

FIG. 7 shows the profile curve of the cam of FIG. 1 when viewed in the direction V1.

FIG. 8 is an exploded view showing the orthotic of FIG. 1.

FIG. 9 is an enlarged front view showing a portion 7′ of FIG. 1.

FIG. 10 is a partial front view showing an orthotic according to one embodiment of the disclosure.

FIG. 11 is a partially exploded view showing an orthotic according to another embodiment of the disclosure.

### DETAILED DESCRIPTION OF THE DISCLOSURE

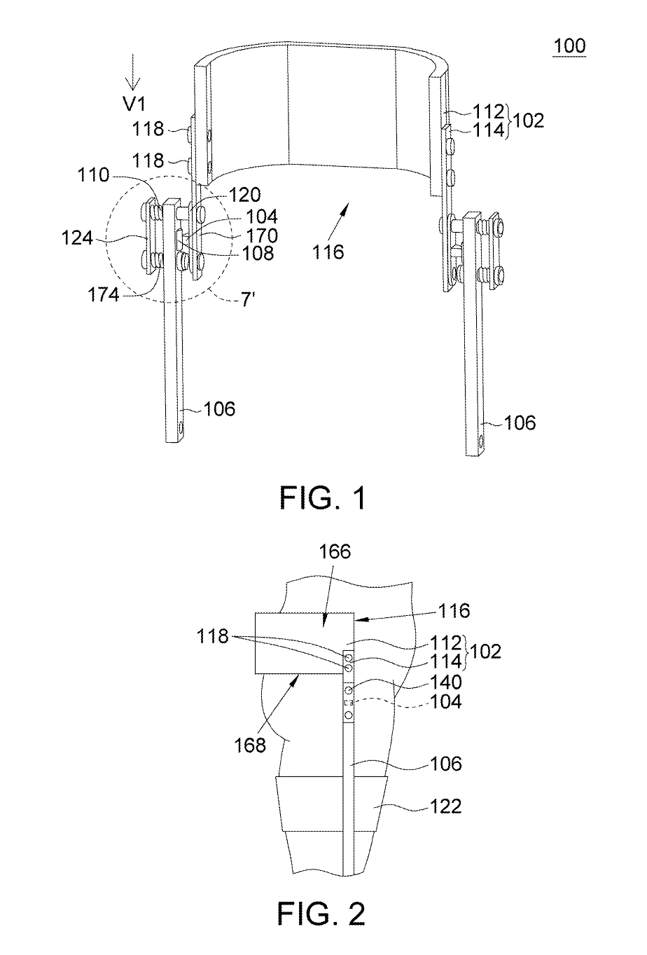

FIG. 1 is an assembled view showing an orthotic 100 according to a preferred embodiment of the disclosure. Referring to FIG. 1, the orthotic 100 for correcting a movement of a hip joint of a user includes a support frame 102 and two assistive correcting mechanisms. The assistive correcting mechanisms are symmetrically disposed on two sides of the support frame 102. Each assistive correcting mechanism includes a cam 104, a leg fixer 106, a following element 108, a pressing plate 124 and restoring elements 110 and 174. The restoring elements 110 and 174 are springs, for example. The following element 108 is a roller, for example. The cam 104 and the following element 108 constitute a cam movement mechanism. However, this does not intend to restrict the disclosure. In other aspects, the following element 108 may also be a ball, a roller or any other following element, which can form a kinematic pair together with the cam 104.

FIG. 2 is a schematic illustration showing the orthotic 100 of FIG. 1 attached to a patient. Referring to FIG. 2, the support frame 102 includes a shielding portion 112 and a cam disposing portion 114. The shielding portion 112 shields the buttock or waist of the patient. The shielding portion 112 is disposed on the waist of the patient with an opening 116 facing the backside or front side of the patient. In this embodiment, the illustrated shielding portion 112 has the opening 116 facing the front side of the patient. That is, the left-side leg fixer 106 of FIG. 1 is fixed to the right leg of the patient.

The leg fixer 106 includes a leg shield 122 for shielding the leg of the patient to fix the leg fixer 106 to the leg of the patient so that the movement of the leg of the patient can move the leg fixer 106.

The cam disposing portion 114 is a link fixedly disposed on a side surface 166 of shielding portion 112 through bolts 118. A portion 170 of the cam disposing portion 114 is extends in a direction toward a bottom surface 168 of the shielding portion 112 connected to the side surface 166. That is, the portion 170 of the cam disposing portion 114 extends in an extending direction of the leg of the patient. The leg fixer 106 is pivotally connected to the portion 170 of the cam disposing portion 114. Thus, the position of the cam 104 may be disposed substantially adjacent to the hip joint of the patient.

In addition, the support frame 102 includes a cam plate 120 (shown in FIG. 1) fixedly disposed on the cam disposing portion 114, wherein the cam 104 is fixedly disposed to the cam plate 120.

The movement between the cam 104 and the following element 108 may make the walking gait of the patient satisfy the normal gait curve. In detail, when the leg of the patient is moving the leg fixer 106, a predetermined relative movement between the cam 104 and the following element 108 is generated to correct the movement range of the hip joint. The predetermined relative movement is determined according to one movement range of the movements of flexion, extension, abduction and adduction of the walking gait. The movement range satisfies the normal gait curve. The orthotic 100 of this embodiment simultaneously provides the degrees of freedom of the movements of flexion, extension, adduction and abduction, so the patient may obtain the opportune correcting procedure closer to the normal gait curve.

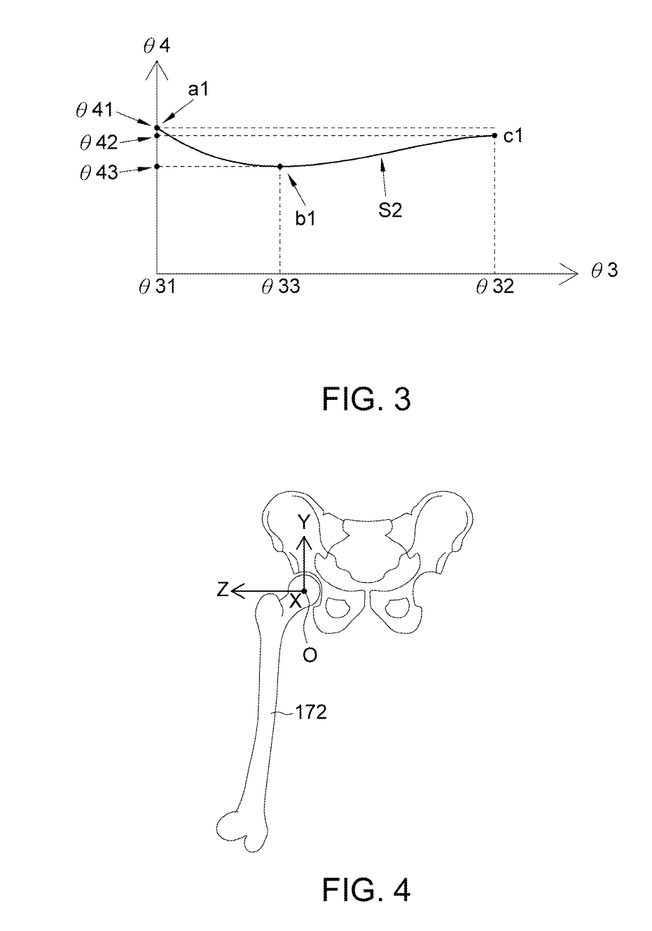

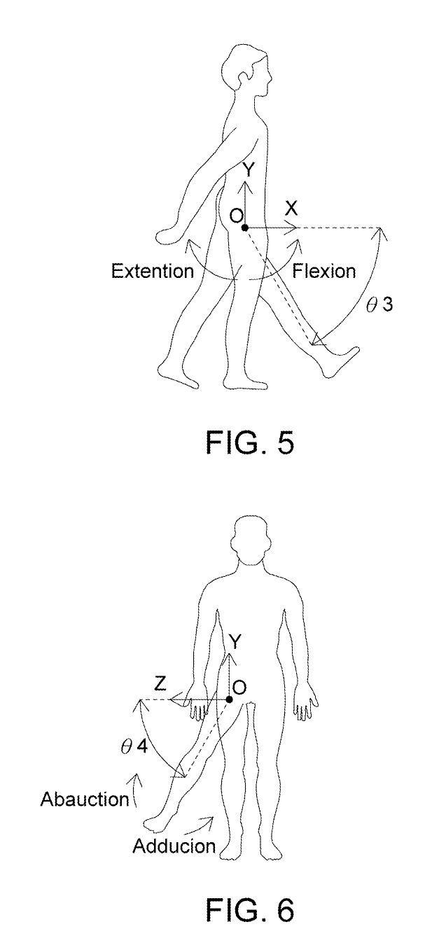

The detailed descriptions will be made with reference to FIGS. 3 to 6. FIG. 3 shows the normal gait curve. FIG. 4 shows the coordinate definition of the gait curve of FIG. 3. FIG. 5 shows the movement of flexion and extension. FIG. 6 shows the movement of abduction and adduction. As shown in FIG. 3 and FIG. 4, the angle θ3 defined in FIG. 3 is the included angle between the +X-axis and the right leg bone 172, which rotates about the Z-axis with the fulcrum O serving as the center, that is, the angle occurred when the right leg bone 172 has the movements of flexion and extension. As shown in FIG. 3 and FIG. 6, the angle θ4 defined in FIG. 3 is the included angle between the +Z-axis and the right leg bone 172, which rotates about the X-axis with the fulcrum O serving as the center, that is, the angle occurred when the right leg bone 172 has the movements of abduction and adduction. The fulcrum O is the original in the coordinate system of FIG. 4.

In the normal gait curve, as shown in FIG. 3, when the right foot starts to step forward, the right leg bone 172 simultaneously has the movements of flexion and abduction. When the heel of the right foot is separated form the ground, the included angle between the right leg bone 172 and the +X-axis (see the coordinate system of FIG. 5) is the angle θ31, and the included angle between the right leg bone 172 and the +Z-axis (see the coordinate system of FIG. 6) is the angle θ41. When the right foot continuously walks until the included angle between the right leg bone 172 and the +X-axis reaches the angle θ33, the right leg bone 172 starts to perform the movements of flexion and adduction simultaneously. Wherein, the point b1 of FIG. 3 (the angle θ33 and θ43) is the limit position of the right leg bone 172 during the movement. When the included angle between the right leg bone 172 and the +X-axis reaches the angle θ32, the normal gait curve S1 of FIG. 3 is completed. At this time, the included angle between the right leg bone 172 and the +Z-axis is the angle θ42.

The movements of the left leg bone (not shown) and the right leg bone 172 are symmetrical, so detailed descriptions of the movements of the left leg bone will be omitted.

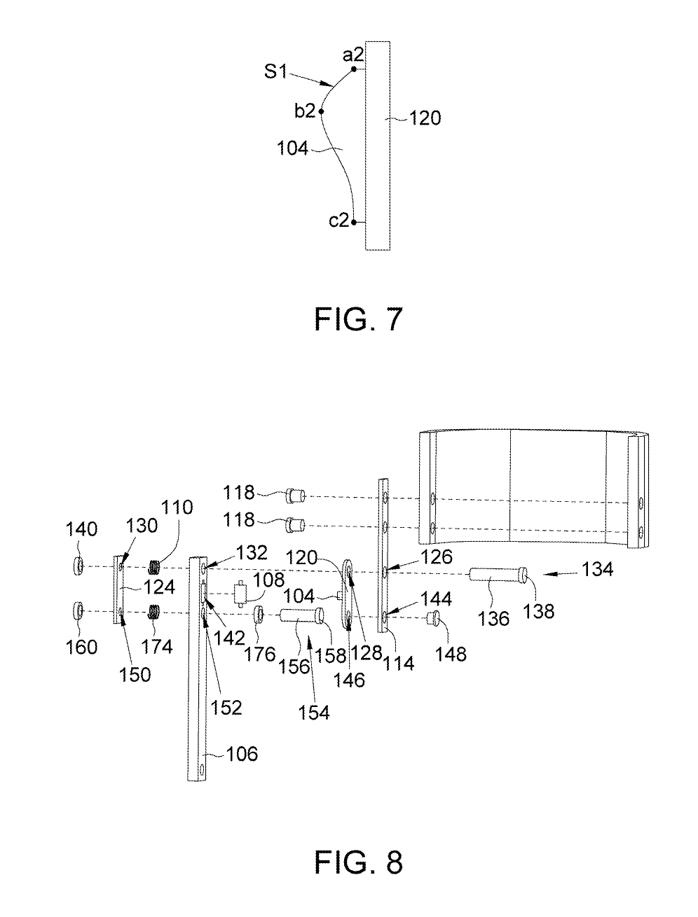

FIG. 7 shows the profile curve of the cam of FIG. 1 when viewed in the direction V1. The profile curve S1 of the cam 104 of FIG. 7 is obtained by performing a cam profile design according to the normal gait curve S2 of FIG. 3. The point a1 of FIG. 3 corresponds to the point a2 of FIG. 7, the point b1 of FIG. 3 corresponds to the point b2 of FIG. 7, and the point c1 of FIG. 3 corresponds to the point c2 of FIG. 7. However, this does not intend to restrict the disclosure. Different the profile of cam is obtained according to different movement curve.

The cam mechanism constituted by the following element 108 and the cam 104 can restrict the movement of the leg bone to satisfy the normal gait curve. The detailed structure of the correcting mechanism of this embodiment will be described in the following.

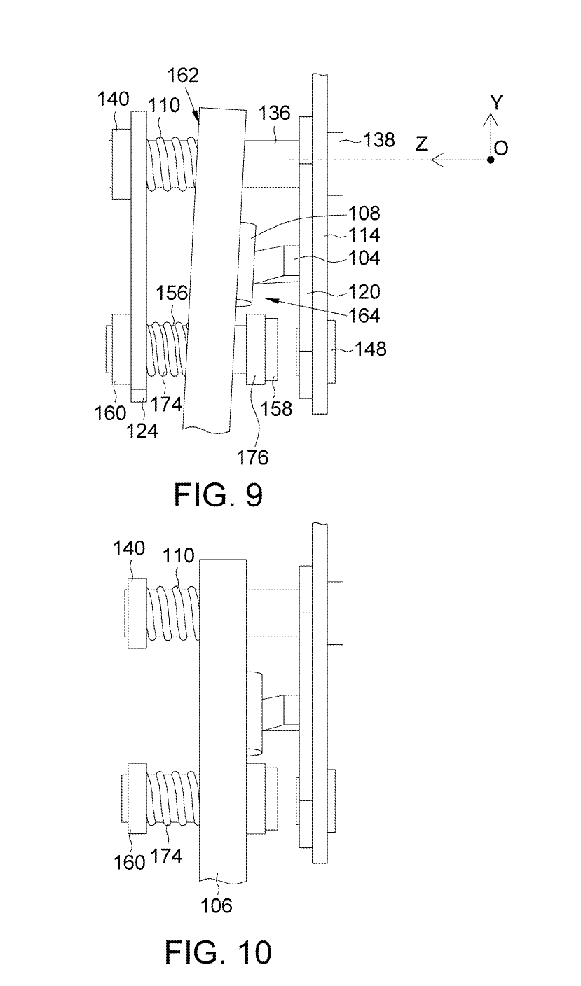

FIG. 8 is an exploded view showing the orthotic of FIG. 1. Referring to FIG. 8, the leg fixer 106 has a first pivot hole 132, and the cam plate 120 has a second pivot hole 128. The pressing plate 124 has a third pivot hole 130, and the cam disposing portion 114 has a fourth pivot hole 126. The support frame 102 further includes a pivot element 134, the position of which substantially corresponds to the hip joint of the patient. The pivot element 134 includes a first rod 136, a first stopper portion 138 and a second stopper portion 140. The first rod 136 is passed through the fourth pivot hole 126, the second pivot hole 128, the first pivot hole 132, the restoring element 110 and the third pivot hole 130. The first stopper portion 138 and the second stopper portion 140 are fixedly connected to the first rod 136. FIG. 9 is an enlarged front view showing a portion 7′ of FIG. 1. As shown in FIG. 9, the cam disposing portion 114 of the support frame 102, the cam plate 120, the leg fixer 106, the restoring element 110 and the pressing plate 124 are restricted between the first stopper portion 138 and the second stopper portion 140, and thus cannot escape from each other to lose the mechanism movement function.

In this embodiment, as shown in FIG. 8, the first rod 136 of the pivot element 134 has threads, the combination of the first rod 136 and the first stopper portion 138 is a bolt, and the second stopper portion 140 is a nut. The combination of the bolt and the nut provides a removable fixing manner but does not intend to restrict the disclosure. In other aspects, the first rod 136 may be a rivet, and the first stopper portion 138 and the second stopper portion 140 are rivet heads formed by two ends of the rivet after the rivet is riveted to the cam disposing portion 114 and the pressing plate 124.

Referring back to FIG. 8, the leg fixer 106 further has a fourth penetrating hole 152 and a slot 142. The slot 142 is substantially located between the fourth penetrating hole 152 and the first pivot hole 132. The following element 108 is rotatably disposed in the slot 142 and is to be connected to the cam 104 to form a cam kinematic pair together with the cam 104.

The cam disposing portion 114 of the support frame 102 further has a first penetrating hole 144, and the cam plate 120 further has a second penetrating hole 146. The cam 104 is disposed between the second pivot hole 128 and the second penetrating hole 146. The orthotic 100 further includes a fixing member 148, which is passed through the first penetrating hole 144 and the second penetrating hole 146 to fix the cam plate 120 to the cam disposing portion 114. Herein, the fixing member 148 is, for example, a bolt, which does not intend to restrict the disclosure. In other aspect, the cam plate 120 may be fixedly disposed on the cam disposing portion 114 by way of welding.

In other aspects, however, the cam plate 120 may also be omitted from the orthotic 100, and the cam 104 is directly fixedly disposed to the cam disposing portion 114.

The pressing plate 124 further has a third penetrating hole 150, and the orthotic 100 further includes a restricting element 154, which includes a second rod 156, a third stopper portion 158 and a fourth stopper portion 160. The second rod 156 is passed through a washer 176, the fourth penetrating hole 152, the restoring element 174 and the third penetrating hole 150. The third stopper portion 158 and the fourth stopper portion 160 are fixedly connected to the second rod 156. The pressing plate 124, the restoring element 174 and the leg fixer 106 are restricted between the third stopper portion 158 and the fourth stopper portion 160, as shown in FIG. 9. Thus, the pressing plate 124, the restoring element 174 and the leg fixer 106 cannot escape from one another to lose the mechanism movement function.

In this embodiment, the second rod 156 of the restricting element 154 has threads, the combination of the second rod 156 and the third stopper portion 158 is a bolt, and the fourth stopper portion 160 is a nut. However, this does not intend to restrict the disclosure. In other aspects, the second rod 156 may be a rivet, and the third stopper portion 158 and the fourth stopper portion 160 may be rivet heads formed by two ends of the rivet after the rivet is riveted to the leg fixer 106 and the pressing plate 124.

As shown in FIG. 9, the restoring elements 110 and 174 connect the pressing plate 124 to the leg fixer 106 and push the leg fixer 106 in a direction toward the cam 104 so that the cam 104 is kept in contact with the following element 108 of the leg fixer 106 during the movement. When the leg fixer 106 is moved, the restoring elements 110 and 174 may provide a force of restoring the leg fixer 106. For example, when the leg of the patient is performing the movement of abduction (see FIG. 9), the restoring elements 110 and 174 store the elastic potential energy. When the leg of the patient enters the movement of adduction from the movement of abduction, the restoring elements 110 and 174 release the elastic potential energy and are thus moved in the adduction direction (the −Z-axis direction of FIG. 6) to push the leg fixer 106 to assist the movement of adduction of the leg.

Since the right leg bone 172 rotates about the X-axis with the fulcrum O serving as the center, the compression of the restoring elements 174 generated by the leg fixer 106 compressing the restoring elements 174 is larger than the compression of the restoring elements 110 generated by the leg fixer 106 compressing the restoring elements 110. However, this does not intend to restrict the disclosure. The compression of the restoring elements 174 and the compression of the restoring elements 110 depends on the profile of the cam 104.

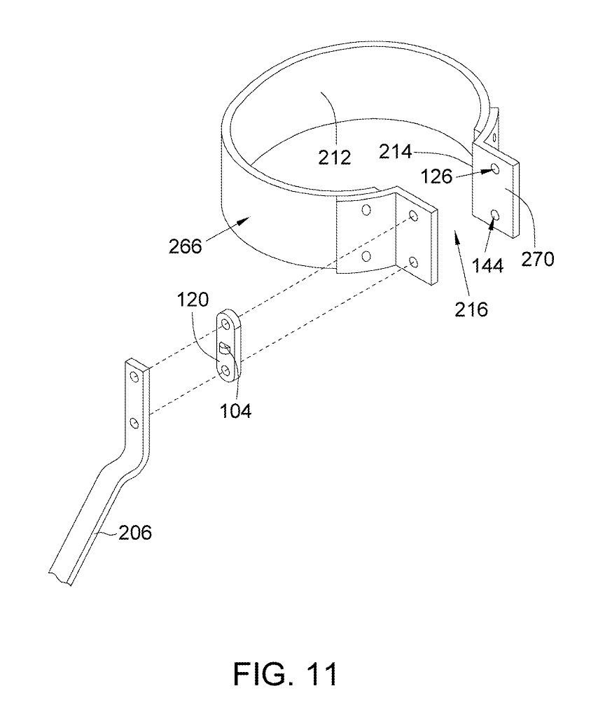

Although the orthotic 100 of this illustrative embodiment includes the pressing plate 124, it does not intend to restrict the disclosure. FIG. 10 is a partially schematic illustration showing an orthotic according to one embodiment of the disclosure. As shown in FIG. 10, the pressing plate 124 is omitted from the orthotic of this embodiment so that the restoring element 110 is connected to the second stopper portion 140 and the leg fixer 106 while the restoring element 174 is connected to the fourth stopper portion 160 and the leg fixer 106.

The cam 104 is located at an arbitrary position between the restoring elements 110 and 174 so that the line contact is held between the following element 108 and the cam 104. When the line contact is held between the following element 108 and the cam 104 during the movement, the cam 104 and the following element 108 generate the more precise relative movement to precisely satisfy the normal gait curve of FIG. 3. In addition, when the contact between the following element 108 and the cam 104 is the line contact, the lifetimes of the following element 108 and the cam 104 are also longer.

Referring also to FIG. 9, preferably but non-restrictively, the cam 104 is substantially located at the middle position between the second pivot hole 128 and the second penetrating hole 146. In this case, the restoring elements 110 and 174 may have the same specification to provide the symmetrical forces to the leg fixer 106.

Although the position of the cam disposing portion 114 of this embodiment substantially corresponds to the neighboring portion (see FIG. 2) of the hip joint of the patient, this does not intend to restrict the disclosure. FIG. 11 is a partially exploded view showing an orthotic according to another embodiment of the disclosure. Referring to FIG. 11, the orthotic of this embodiment includes a shielding portion 212, a cam disposing portion 214, a cam plate 120 and a leg fixer 206. The assembling method of the leg fixer 206 is similar to that of the leg fixer 106 of FIG. 8, so detailed descriptions thereof will be omitted.

The shielding portion 212 has an opening 216 for exposing the navel of the patient. The cam disposing portion 214 is disposed adjacent to the opening 216 and on a side surface 266 of the shielding portion 212. The portion of the cam plate 120, which is disposed in the cam disposing portion 214 and overlapped with the opening 216, is a bent portion 270 so that the position of the cam 104 substantially corresponds to the navel of the patient. However, as long as the dimensions of the opening 216 of the shielding portion 212 and the dimensions of the cam disposing portion 214 are properly modified, the cam 104 may be substantially disposed on two sides of the navel of the patient and approach the position of the waist.

When the position of the cam 104 is substantially located on the waist or navel of the patient, the patient can see the cam mechanism when the patient bends his/her head without the lateral rotation of his/her head. Thus, a more convenient and laborsaving operation mode may be provided to the patient.

The orthotic according to each embodiment of the disclosure can simultaneously provide the degrees of freedom of the movements of flexion, extension, adduction and abduction in the walking gait to the patient so that the patient obtains the opportune correcting procedure closer to the normal gait curve.

While the disclosure has been described by way of example and in terms of preferred embodiments, it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. An orthotic for correcting a movement of a hip joint of a user, the orthotic comprising:
a support frame to be attached to the user;
a cam fixed to the support frame;
a leg fixer pivotally connected to the support frame;
a following element moveably mounted on the leg fixer and connected to the cam; and
a restoring element, connected to the leg fixer, for providing a force of restoring the leg fixer when the leg fixer is moved;
wherein when the leg fixer is being moved, a predetermined relative movement between the cam and the following element is generated to correct a movement range of the hip joint.

2. The orthotic according to claim 1, wherein the predetermined relative movement is determined by a movement range of flexion, extension, abduction and adduction.

3. The orthotic according to claim 2, wherein the movement range satisfies a normal gait curve.

4. The orthotic according to claim 1, wherein the leg fixer has a first pivot hole, and the support frame comprises:
a cam plate having a second pivot hole, wherein the cam is disposed to the cam plate; and
a pivot element, passing through the first pivot hole and the second pivot hole, for pivotally connecting the cam plate to the leg fixer.

5. The orthotic according to claim 4, wherein the support frame has a first penetrating hole, the cam plate further has a second penetrating hole, the cam is disposed between the second pivot hole and the second penetrating hole, and the orthotic further comprises:
a fixing member, passing through the first penetrating hole and the second penetrating hole, for fixing the cam plate.

6. The orthotic according to claim 4, wherein the pivot element comprises:
a first rod passing through the first pivot hole, the restoring element and the second pivot hole; and
a first stopper portion and a second stopper portion both connected to on the first rod;
wherein the support frame, the restoring element, the cam plate and the leg fixer are restricted between the first stopper portion and the second stopper portion.

7. The orthotic according to claim 6, wherein the first rod has threads, a combination of the first rod and the first stopper portion is a bolt, and the second stopper portion is a nut.

8. The orthotic according to claim 6, further comprising:
a pressing plate having a third pivot hole, wherein the first rod further is passed through the third pivot hole to restrict the pressing plate between the first stopper portion and the second stopper portion;
wherein the restoring element connects the pressing plate to the leg fixer.

9. The orthotic according to claim 1, wherein the leg fixer further has a fourth penetrating hole, and the orthotic further comprises a restricting element, which comprises:
a second rod passing through the fourth penetrating hole and the restoring element; and
a third stopper portion and a fourth stopper portion both connected to the second rod;
wherein the leg fixer and the restoring element are restricted between the third stopper portion and the fourth stopper portion.

10. The orthotic according to claim 9, further comprising:
a pressing plate having a third penetrating hole, wherein the second rod further is passed through the third penetrating hole to restrict the pressing plate between the third stopper portion and the fourth stopper portion.

11. The orthotic according to claim 11, wherein the second rod has threads, a combination of the second rod and the third stopper portion is a bolt, and the fourth stopper portion is a nut.

12. The orthotic according to claim 1, wherein the following element is a roller.

13. The orthotic according to claim 1, wherein the leg fixer comprises a leg shield for shielding a leg of the user to fix the leg fixer to the leg of the user.

14. The orthotic according to claim 1, wherein the support frame comprises a shielding portion and a cam disposing portion, the cam disposing portion is disposed to the shielding portion, the shielding portion is used shielding the user, and the cam is disposed to the cam disposing portion.

15. The orthotic according to claim 14, wherein the cam disposing portion is disposed to a side surface of the shielding portion, a portion of the cam disposing portion is extended along a direction toward a bottom surface of the shielding portion, and the cam is disposed to the portion of the cam disposing portion.

16. The orthotic according to claim 15, wherein the portion of the cam disposing portion is extended, along the direction toward the bottom surface, to a side surface of the leg of the user.

17. The orthotic according to claim 14, wherein the shielding portion has an opening for exposing a navel of the user, the cam disposing portion is disposed adjacent to the opening and on the shielding portion, and the cam is disposed on an overlapped portion between the cam disposing portion and the opening.
